(19) United States
(12) Patent Application Publication
(10) Pub. No.: US 2015/0216551 A1
(43) Pub. Date: Aug. 6, 2015

(54) Ultrasound Surgical Micromotor

(71) Applicant: SOCIETE POUR LA CONCEPTION DES APPLICATIONS DES TECHNIQUES ELECTRONIQUES, Merignac (FR)

(72) Inventors: Francis Dieras, Bordeaux (FR); Pascal Regere, Blanquefort (FR)

(21) Appl. No.: 14/415,032

(22) PCT Filed: Jul. 12, 2013
(86) PCT No.: PCT/FR2013/051670

§ 371 Date: Jan. 15, 2015

(30) Foreign Application Priority Data
Jul. 16, 2012 (FR) 1256861

(51) Int. Cl. A61B 17/32

(52) U.S. Cl. CPC A61B 17/320092; A61B 17/1624

(57) ABSTRACT

A surgical handpiece includes a housing for receiving an insert, and an ultrasound piezoelectric transducer having a piezoelectric motor placed between a counterweight and an amplifier portion. The piezoelectric transducer is suitable for transmitting ultrasound waves from a distal end of the amplifier portion. The distal end is present in the housing, and rotary drive means is present in the housing. The rotary drive means are movable independently of the ultrasound piezoelectric transducer, and the transducer is static relative to the handpiece.

### BACKGROUND OF THE INVENTION

The present invention relates to surgical treatment appliances and, more particularly, to surgical handpieces for treating biological tissues (enamel, dentine, mucous membrane, bone, . . . ) or implanted prosthesis such as implants, prostheses, or any other active implantable device (heart valve, . . . ).

The invention applies more particularly to cutting and/or drilling into biological tissues of various densities situated close to anatomical structures that are fragile and that need to be preserved, such as a membrane or a vital organ, for example.

In conventional manner, for example when practicing osteotomy (e.g. cutting away bone from a knee), it is general practice to use a surgical handpiece provided with a micromotor driving a rotary cutter, the rotary motion of the cutter serving to dig into the bone tissue in highly effective manner. The same type of surgical handpiece is preferably used when it is desired to drill or alter a cavity that is to receive a dental or an orthopedic implant.

That operating technique is relatively effective, but nevertheless involves certain risks, in particular because of the speed of rotation of the cutter which is sometimes difficult to control, and can thus lead to irreversible lesions of neighboring tissues such as a nerve or an artery. When drilling for an implant, for example, the depth of the drilling must be monitored particularly carefully to avoid harming the underlying tissues. When fitting dental implants, it is important for example to avoid harming the nerve that passes through the mandible or the sinus membrane when drilling the jawbone.

To overcome those drawbacks, initial solutions have been proposed. By way of example, patent application EP 1 235 527 describes an instrument for putting dental implants into place. That instrument is provided with a device for monitoring both the depth and the angle of the drilling while a dental implant is being put into place.

Patent application EP 1 733 694 also describes a rotary surgical handpiece with a mechanism for regulating pressure.

Nevertheless, those technological solutions are not always satisfactory in terms of effectiveness in drilling or cutting.

In known manner, the use of ultrasound in certain surgical handpieces presents advantages for those interventions. That technique makes it possible to replace the dangerous rotary motion of the micromotor with ultrasound vibration that is transmitted from the piezoelectric transducer to a sonotrode of surface and shape that enable bone to be cut effectively, while being safer. The operation of cutting tissue or of drilling is more accurate and better controlled by the operator. On contact with tissue of lower density, the action of the tool becomes less contusive, thereby considerably diminishing the danger of the action on the anatomical structures that are to be preserved.

FIG. 1 shows an example of a control device 100 that comprises an ultrasound generator 110 connected to a surgical handpiece 115 by a cord 111. A sonotrode or ultrasound insert 130 is mounted on the top or distal portion of the handpiece 115. In well-known manner, the handpiece 115 has a transducer (not shown) made of a piezoelectric material and mechanically coupled to the insert 130 so as to transmit ultrasound vibration thereto of amplitude that is a function of the power delivered by the ultrasound generator.

Ultrasound devices of that type are used in various fields of surgery, including ophthalmology for phaco-emulsification of the lens, neurosurgery, or indeed orthopedics.

A drilling device for surgical use is also disclosed in U.S. Pat. No. 6,204,592 B1 (cf. FIG. 1 of that document). That drilling device has both an ultrasound piezoelectric transducer and a rotary drive micromotor.

The piezoelectric transducer is for generating vibratory impulses that are transmitted to an insert mounted at the distal end of the handpiece in order to impart percussive axial motion on the insert. The micromotor serves to rotate an assembly including a rotary shaft (“horn 16”). An instrument is positioned at the distal end of that rotary shaft and the piezoelectric transducer is secured to the other end (i.e. the proximal end) of the rotary shaft.

That type of handpiece thus advantageously combines both vibratory and rotary motions so as to optimize the action of the insert on the biological tissue to be treated.

The solution proposed in document U.S. Pat. No. 6,204,592 B1 nevertheless presents major drawbacks in terms in particular of the complexity of the structure of the handpiece. The rotation and the vibration to which the insert is subjected are also relatively difficult to control when operating with that type of surgical handpiece.

There therefore exists a need at present for a surgical handpiece that provides a better compromise between effectiveness in cutting and/or drilling, control over the action of the tool on the biological tissues to be treated, and simplicity of fabrication.

### OBJECT AND SUMMARY OF THE INVENTION

To this end, the present invention provides a surgical handpiece comprising:

a housing for receiving an insert;

an ultrasound piezoelectric transducer comprising a piezoelectric motor placed between a counterweight and a proximal end of an amplifier portion, the piezoelectric transducer being suitable for transmitting ultrasound waves from a distal end of said amplifier portion to the insert, the distal end being present in the housing; and

rotary drive means present in said housing for driving in rotation the insert;

wherein the rotary drive means are movable independently of the ultrasound piezoelectric transducer, said ultrasound piezoelectric transducer being static relative to the handpiece.

Thus, the ultrasound piezoelectric transducer remains static relative to the rotary drive means while the rotary drive means are in operation.

Integrating rotary drive technology with ultrasound vibration technology makes it possible to offer effectiveness that is optimized in terms of cutting and/or drilling, for example. The operator can advantageously use a single handpiece for performing all of the treatments needed on the tissues concerned, thereby avoiding the need to pass from one appliance to another during the intervention.

Advantageously, the invention makes it possible both to optimize the adjustment of the amplitude of the ultrasound vibration, and to optimize the adjustment of the speed of rotation imparted by the rotary drive means either simultaneously or independently.

The invention also makes it possible to optimize the transmission of ultrasound vibration without disturbance due to the rotation, if any, of the rotary drive means.

By configuring the rotary drive means to move independently of the ultrasound piezoelectric transducer and by configuring the piezoelectric transducer to be static relative to the handpiece, the invention thus makes it possible to improve control over the ultrasound vibration generated by the piezoelectric transducer. Furthermore, the lifetime of the piezoelectric transducer is significantly increased thereby.

In addition, the structure of the handpiece of the invention is relatively simple and presents robustness that is increased compared with other devices such as that envisaged in above-commented document U.S. Pat. No. 6,204,592 B1, for example.

The energy needed for driving the insert in rotation is also limited because none of the elements of the ultrasound piezoelectric transducer rotates with the rotary drive means.

The invention thus applies most particularly to cutting and/or drilling biological tissue of various densities situated close to fragile anatomical structures that need to be preserved such as a membrane, a nerve, or a vital organ, for example. This applies for example during laminectomies in spinal surgery, which consist in removing one or more vertebral laminae close to the spinal chord or to the base of the cranial nerves.

In a first embodiment, the rotary drive means comprise a micromotor connected to the housing via a transmission shaft.

In a first variant, the axis of rotation of the rotary drive means is perpendicular to a distal portion of the transmission shaft. This configuration serves to give the handpiece an angled appearance, which is more ergonomic for the operator when performing certain types of intervention in which access to the zone for treatment is difficult.

In a second variant, the axis of rotation of the rotary drive means in the housing is parallel to a distal portion of the transmission shaft. This straight configuration of the handpiece may be particularly suitable for certain types of intervention, such as for treating the vertebral column, for example.

This second variant is likewise particularly suitable for operating on Chiari's malformation in pediatric surgery when a luxating dysplasia of the hip requires osteotomy of the pelvis to receive the head of the femur. The operating field is characterized by being very constrained since the sciatic nerve and the gluteal artery are located at the end of the osteotomy path, thus requiring great precautions to be taken during the operation.

The innocuousness of ultrasound osteotomy has also been demonstrated by experiments where anatomo-pathological observation does not reveal cellular lesions in the proximity of the cut, whether on the periosteum, the endosteum, osteocyte cells, or cells present in bone vascularization.

Still in this second variant, the ultrasound piezoelectric transducer may be configured to transmit ultrasound waves to said housing via the rotary drive means.

In a second embodiment, the rotary drive means comprise an air turbine and a transmission duct, the turbine being suitable for entering into rotation under the action of a stream of gas (e.g. air) delivered via said transmission duct.

In a first variant of this second embodiment, the axis of rotation of the rotary drive means in the housing is perpendicular to a distal portion of the transmission duct. As mentioned above, this configuration makes it possible to give the handpiece an angled appearance, thereby making it more ergonomic for the operator in certain types of intervention in which access to the zone for treatment is difficult.

In a second variant of this second embodiment, the axis of rotation of the rotary drive means in the housing is parallel to a distal portion of the transmission duct. This straight configuration of the handpiece is likewise particularly suitable for certain types of intervention, such as for treating the vertebral column, for example.

In a particular embodiment of the invention, the ultrasound piezoelectric transducer includes a ceramic vibratory transmission element arranged at the distal end of the amplifier portion so as to transmit the ultrasound waves in the housing.

In a particular embodiment, the amplifier portion presents a constriction of position along the amplifier portion that is determined in such a manner as to maximize the amplitude of the vibratory motion of the distal end of the amplifier portion.

In a particular embodiment, the handpiece of the invention further includes an insert placed in part in the housing so as to be capable of co-operating with the ultrasound piezoelectric transducer and with the rotary drive means.

In preferred manner, in the working position, the insert is arranged in such a manner that its proximal end is in contact with the distal end of the amplifier portion, and that the body of the insert is capable of being driven in rotation by the rotary drive means.

### BRIEF DESCRIPTION OF THE DRAWINGS

Other characteristics and advantages of the present invention appear from the following description made with reference to the accompanying drawings, which show an embodiment having no limiting character. In the figures:

FIG. 1, described above, is a diagram of an example of a known control device of the prior art;

FIG. 2 is a diagrammatic longitudinal section view of a surgical handpiece in a first embodiment of the invention;

FIG. 3 is a diagrammatic plan view of the FIG. 2 handpiece;

FIGS. 4A and 4B show variant shapes for the amplifying portion of the ultrasound piezoelectric transducer shown in FIG. 2;

FIG. 5 is a diagrammatic longitudinal section view of a surgical handpiece in a second embodiment of the invention;

FIGS. 6 and 7 are section views of variants of the handpieces of FIGS. 2 and 5 respectively;

FIG. 8 is a section view of another variant of the surgical handpiece of FIG. 2; and

FIGS. 9A to 9I are diagrammatic views of example inserts that may be fitted to the handpiece of the invention.

### DETAILED DESCRIPTION OF EMBODIMENTS OF THE INVENTION

The present invention relates to a surgical handpiece for treating (cutting, drilling, . . . ) biological tissues of different densities by means of an instrument (or an insert).

In order to mitigate the drawbacks of prior art devices, the handpiece of the invention advantageously incorporates two distinct technologies: ultrasonic vibration of the insert in order to important percussive motion thereto together with rotary drive of the insert with the help of suitable rotary drive means.

This handpiece is thus suitable for imparting both rotary motion and percussive motion to the cutting and/or drilling tool (i.e. the insert) and it can do so either in alternation or simultaneously.

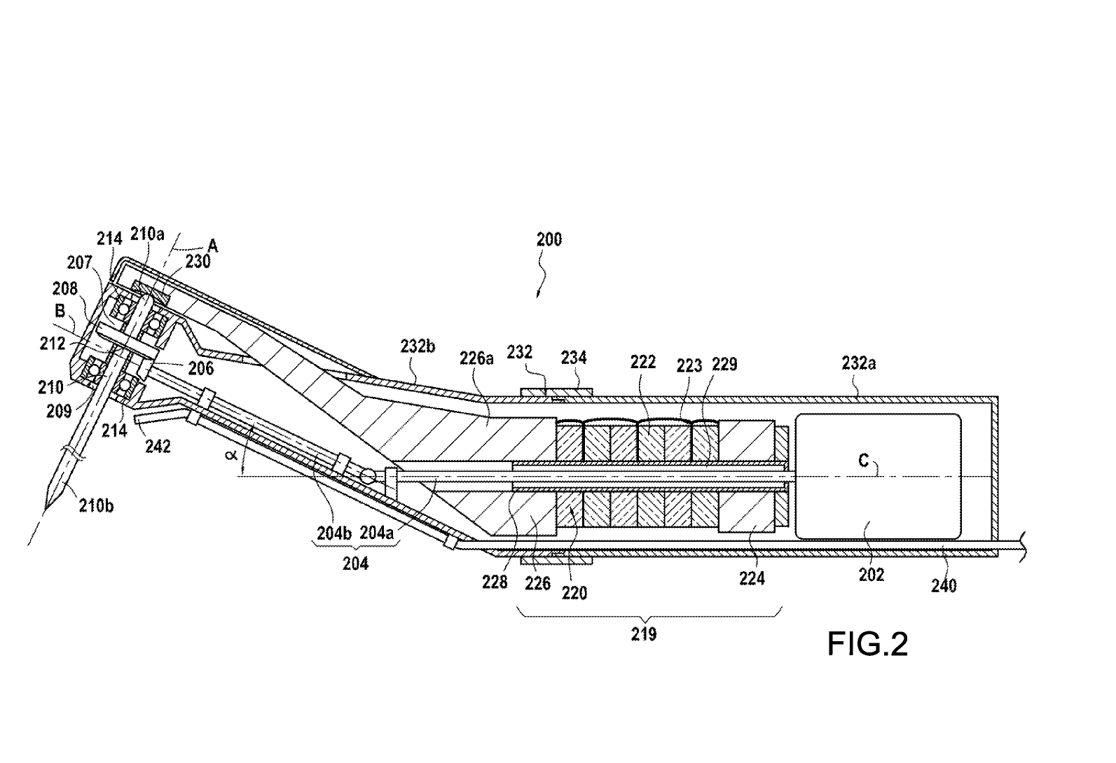

The surgical handpiece 200 in a first embodiment of the invention is described below with reference to FIGS. 2, 3, 4A, and 4B.

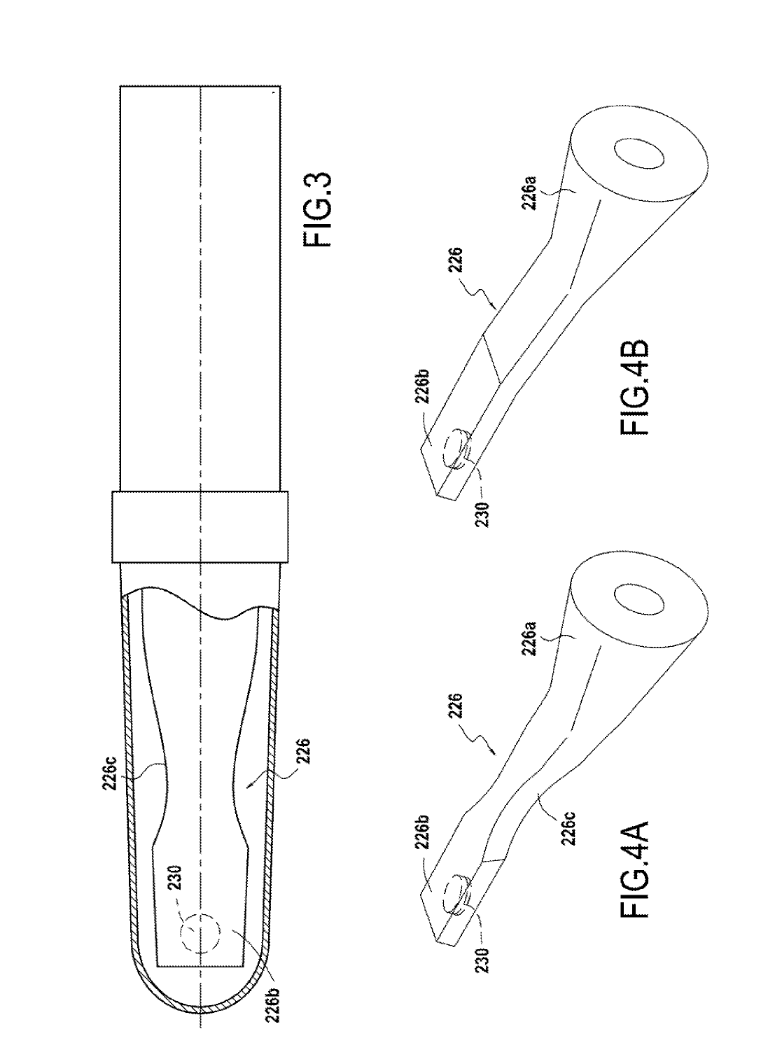

More specifically, FIG. 2 is a diagrammatic lateral section view of the surgical handpiece 200. FIG. 3 is a plan view of the same handpiece 200.

The surgical handpiece 200 includes a micromotor 202 that is situated in the proximal portion of the handpiece. The micromotor 202 is mechanically coupled to the proximal portion 204a of a transmission shaft 204 that extends inside the body of the handpiece 202.

In this example, the transmission shaft 204 also includes a distal portion 204b that is mechanically coupled to the proximal portion 204a by a universal joint.

The distal portion 204b of the transmission shaft 204 in this particular example presents a non-zero angle α relative to the proximal portion 204a (angle between the axes of rotation B and C in FIG. 2). As described in greater detail below, this angle α may be zero in other embodiments of the invention.

In this example, the head of the distal portion 204b of the transmission shaft 204 has a gearwheel 206 that co-operates with an annular gear 208, the gearwheel 206 and the annular gear 208 being perpendicular to each other in this example.

The annular gear 208 is located in a housing 207, which housing is arranged to be capable of receiving an insert 210 for treating (cutting, drilling, etc.) biological tissues. The annular gear 208 in this example has a fastener device 212 enabling an appropriate insert 210 to be fastened in the housing 207, depending on the treatment that is to be performed.

In this example, the housing 207 has a cavity situated at the distal end of the handpiece 200 and that opens out to the outside of the handpiece via an orifice 209. In particular, the housing 207 has ball bearings 214 enabling the insert 210 to rotate freely about the axis of rotation A in the housing 207 when the insert is driven in rotation via the transmission shaft 204 by the micromotor 202.

In this example, the assembly that comprises the micromotor 202, the transmission shaft 204, and the annular gear 208 constitutes an example of rotary drive means in the meaning of the invention. Nevertheless, it will be understood that this is merely an exemplary embodiment, and other variants can be envisaged in the ambit of the invention, some of which are described below.

The surgical handpiece 200 also has an ultrasound piezoelectric transducer 219 situated in this example between the micromotor 202 and the distal end of the handpiece 200.

The ultrasound piezoelectric transducer 219 comprises a piezoelectric motor 220, a counterweight 224, and an amplifier portion 226. More precisely, the piezoelectric motor 220 comprises a plurality of ceramic piezoelectric disks 222 with electrical contacts 223 interposed between them so as to enable the piezoelectric motor 220 to be powered electrically. The stack of piezoelectric disks 222 is mechanically constrained at opposite ends by the amplifier portion 226 (at the distal end) and by the counterweight 224 (at the proximal end).

The amplifier portion 226 and the counterweight 224 in this example are connected together by an axial prestress rod 228 (e.g. being held by a stress-applying nut), the rod itself having a central duct 229 passing therethrough from end to end. The duct 229 thus passes through the counterweight 224, through the piezoelectric ducts 222 (together with their electrical contacts 223), and through part of the amplifier portion 226 so as to allow the transmission shaft 204 to pass freely therethrough.

The piezoelectric motor 220 is suitable for generating ultrasound vibration under the effect of electrical power being applied to the electrical contacts 223. Since the operation and the general configuration of an ultrasound piezoelectric transducer are themselves known, they are not described in greater detail below in this document.

In this example, the ultrasound piezoelectric transducer 219 thus constitutes means for generating ultrasound vibration.

The ultrasound vibration generated by the piezoelectric transducer 219 (when it is actuated) is transmitted to the housing 207 via the amplifier portion 226, and in particular from the distal end 226b of the amplifier portion 226 that is present in the end of the housing 207.

As shown in FIG. 4A, in this example, the amplifier portion 226 presents a non-zero angle α between its distal and proximal portions so as to match the angled shape of the handpiece 200. This angled shape of the handpiece is not always required, but it is particularly suitable for dental type treatments, for example, since it is more ergonomic in achieving access to certain treatment zones.

As shown in FIGS. 3 and 4A, in this example, the amplifier portion 226 presents a constriction 226c that serves to control and optimize the amplitude of the ultrasound vibration to which the distal end 226b is subjected when the piezoelectric motor 220 is in operation. By adjusting the position of this constriction 226c along the amplifier portion 226 (during design and fabrication of the amplifier portion), it is possible for example to maximize the vibratory amplitude of the distal end 226b of the amplifier portion 226. The way in which the constriction is positioned is itself known and is therefore not described in greater detail in this document.

Nevertheless, it will be understood that the presence of such a constriction is not essential, as shown in FIG. 4B which shows a rectilinear amplifier portion 226 by way of example.

Consideration is given below to the situation in which an insert 210 is inserted in the housing 207 via the orifice 209 and is placed in a working position such that the proximal end 210a (i.e. the base) of the insert is in abutment against the distal end 226b of the amplifier portion 226, while the distal end 210b of the insert 210 (i.e. its working end) extends outside the housing 207 and thus outside the handpiece 200.

In the present example, the base 210a of the insert is more precisely in contact with a vibration transmission element 230 arranged facing it in the distal end 226b of the amplifier portion 226. In this example, the vibration transmission element 230 is a ceramic part crimped in the distal end 226b, this part being circular in shape. Other shapes (square, . . . ) could nevertheless be envisaged. Adding this part serves to optimize the transmission of ultrasound vibration to the insert 210.

It should be observed that a resilient return force is applied to the insert 210 in order to keep its end 210a in contact with the vibration transmission element 230. Keeping this contact makes it possible to ensure better transmission of the ultrasound vibration generated by the piezoelectric transducer 219 all the way to the insert. The person skilled in the art will understand that the mechanism (not shown in the figures) that serves to apply this return force may be made in various ways (e.g. with the help of a spring), and it is therefore not described in greater detail in this document.

Nevertheless, it will be understood that the above-described ultrasound piezoelectric transducer 219 constitutes merely one particular embodiment and other variants can be envisaged in the ambit of the invention, some of which are described below.

The surgical handpiece 200 is thus suitable for:

imparting percussive motion to the insert 210 by transmitting ultrasound vibration thereto from the ultrasound piezoelectric transducer of the invention; and

driving the insert 210 in rotation about the axis of rotation A of the housing 207 from the rotary drive means of the invention.

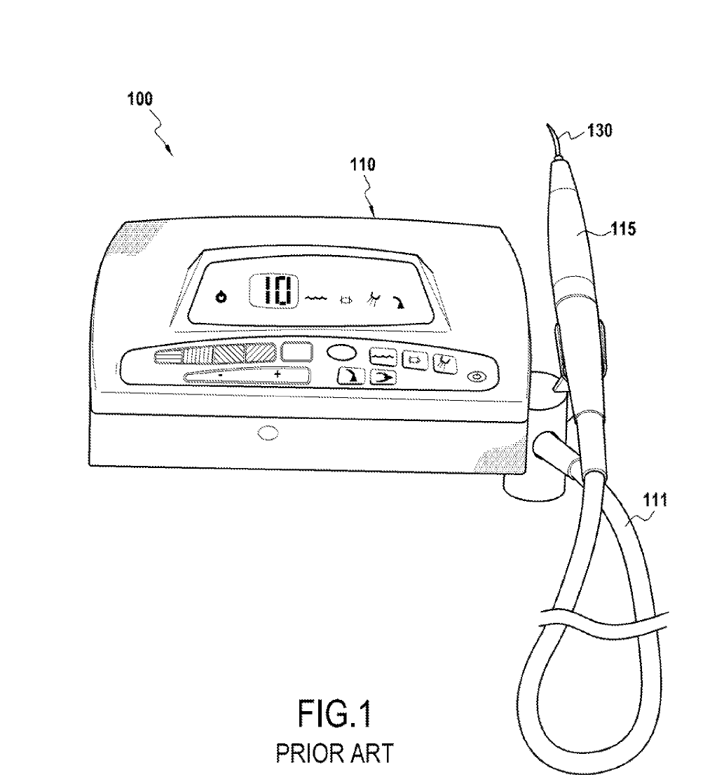

These two actions of rotation and of vibration may be performed simultaneously or in alternation on command of the user, e.g. with the help of a control present on a device of the same type as the control device 100 shown in FIG. 1.

In accordance with the invention, the rotary drive means are movable independently of the ultrasound piezoelectric transducer, the ultrasound piezoelectric transducer being static relative to the handpiece.

Thus, when the transmission shaft 204 is driven in rotation by the micromotor 202, the ultrasound piezoelectric transducer 219 remains static relative to the handpiece 200. This configuration presents major advantages, e.g. relative to handpieces known in the prior art.

Integrating the rotary drive technology and the ultrasound vibration technology serves to obtain effectiveness that is optimized in terms of cutting and/or drilling, for example. With the help of a single handpiece, the operator may advantageously perform all of the treatments needed on the tissues in question, avoiding any need to change from one appliance to another during the intervention.

With reference for example to the device of document U.S. Pat. No. 6,204,592 B1, the piezoelectric transducer and the corresponding electrical connections are secured to the transmission shaft so that the transmission shaft and the piezoelectric transducer rotate together. Consequently, that configuration requires the piezoelectric disk of the piezoelectric motor to be powered electrically via a troublesome system of slip rings (cf. references 54 and 56 in FIG. 2 of that document). That type of annular electrical connection with rubbing is relatively fragile and awkward to assemble, and it can be difficult to adjust.

Furthermore, the configuration proposed in document U.S. Pat. No. 6,204,592 B1 exposes the piezoelectric transducer to high levels of mechanical stress and impact while the transmission shaft is in movement (rotating in the present case), thereby running the risk of reducing the lifetime of the piezoelectric transducer and of degrading the quality of the ultrasound vibration it generates.

By configuring the rotary drive means to move independently of the ultrasound piezoelectric transducer and by configuring the piezoelectric transducer to be static relative to the handpiece, the invention thus makes it possible to improve control over the ultrasound vibration generated by the piezoelectric transducer. Furthermore, the lifetime of the piezoelectric transducer is significantly increased thereby.

The invention also makes it possible to improve control over the rotation of the rotary drive means, and in particular of the transmission shaft 204 in the present example. The consumption of energy (electrical energy in this example) is also reduced because the micromotor does not drive the piezoelectric transducer 220 in rotation.

It is thus possible to adjust both the rotation and the ultrasound vibration in independent manner.

The invention thus applies more particularly to cutting and/or drilling biological tissues of various densities situated in the proximity of fragile anatomical structures that need to be preserved such as a membrane or a vital organ, for example.

The physical properties of the insert 210 are preferably selected in such a manner that the insert can have a contusive action both in rotation and in vibration. By way of example, the insert 210 may be formed with vertical and horizontal ribs, or alternatively with oblique ribs. The ribs may optionally carry diamonds. In a variant, it is possible to use inserts that act in vibration only or in rotation only.

The user is preferably capable of using different inserts depending on the work that is to be performed.

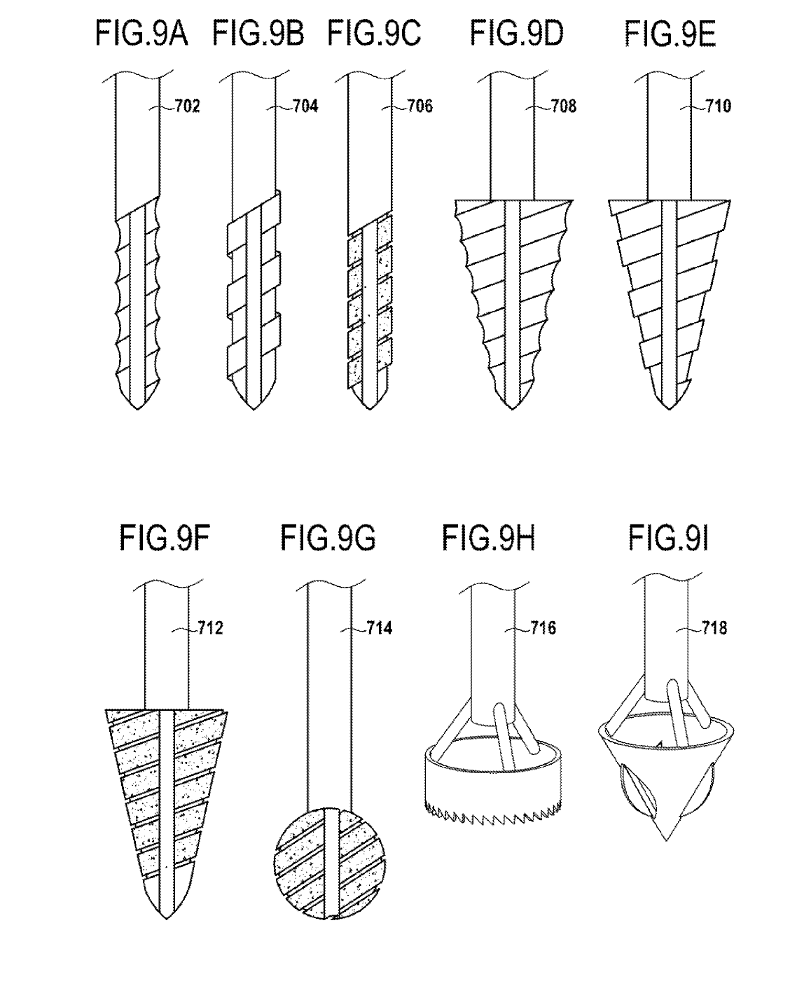

FIGS. 9A to 9I are diagrammatic views showing examples of inserts suitable for being installed in the housing of the handpiece of the invention.

FIGS. 9A, 9B, and 9C show inserts (having respective references 702, 704, and 706) that are in the form of elongate drill bits.

The bits 702 and 704 have respective spiral contusive edges and a spiral contusive blade. The bit 706 carries diamonds and includes grooves that enable tissue residue to be removed. All three of these bits 702, 704, and 706 have a central duct for removing tissue residue.

It should be observed that the inserts 702, 704, and 706 are preferably used for deep osteotomy or drilling operations, e.g. when drilling to install an implant in dental surgery.

The inserts 708, 710, and 712 shown in FIGS. 9D, 9E, and 9F constitute conically-shaped variants of the inserts 702, 704, and 706 respectively.

The insert 714 shown in FIG. 9G carries diamonds and is ball-shaped.

The conically-shaped inserts 708, 710, and 712, and the ball-shaped insert 714 may be used for example in circular osteotomies of varying diameter, in geode-shaped osteotomies for apical resections (e.g. in dental surgery), or they may be used as rasps, e.g. for use on osteophytes or secondary kissing vertebral osteophytes due to an arthritic process.

The insert 716 shown in FIG. 9H is of the trepan or crown saw type and is in the form of a circular ring with saw teeth. This type of insert may be selected for osteotomies that require an accurate bore to fit a prosthetic fastener, for example, or indeed for trepanning the skull in neurosurgery.

The insert 718 of FIG. 9I is another variant of a conical insert, and this example has contusive lugs extending outwards from the surface of the cone. By way of example, this type of insert may be fastened to the end of a catheter for directional arteriectomy (an operation that consists in eliminating an accumulation of atheromatosis from the inside of an artery).

It should also be observed that the internal components of the surgical handpiece 200, and in particular those corresponding to the ultrasound piezoelectric transducer and to the rotary drive means, are enclosed in this example in cladding or a casing 232 comprising a proximal portion 232a and a distal portion 232b.

In this example, the fastening between the proximal and distal portions 232a and 232b of the cladding is reinforced by means of a docking ring 234 at the junction between the cladding portions 232a and 232b.

It should also be observed that the handpiece 200 in this example has an irrigation duct 240 leading to an orifice 242 placed in the proximity of the orifice 209 in the housing 207. This irrigation duct 240 serves to allow a liquid to flow longitudinally in either direction through the handpiece 200 either to irrigate the zone where tissue is being treated, should that be necessary, or alternatively to remove substances (tissue residues, . . . ) that come from the treated zone in question.

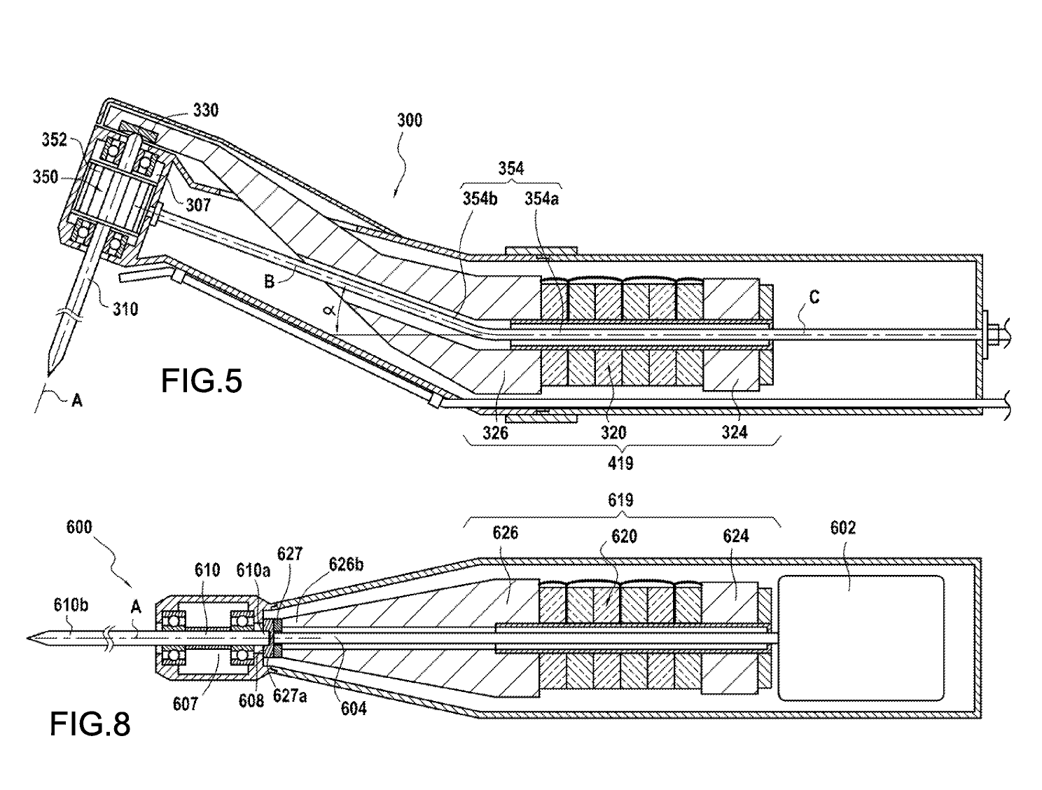

A surgical handpiece 300 in a second embodiment of the invention is described below with reference to FIG. 5.

Most of the components of the handpiece 300 are identical to the corresponding components of the handpiece 200 as described above. Unless otherwise specified, the components of the handpiece 300 present the same properties and perform the same operations as the corresponding components of the handpiece 200.

The ultrasound piezoelectric transducer 319 of the handpiece 300 comprises in particular a piezoelectric motor 320 arranged under pre-stress between a counterweight 324 and an amplifier portion 326, the distal portion of the amplifier portion being present in a housing 307. The elements 319, 320, 324, 326, and 307 are identical respectively to the elements 219, 220, 224, 226, and 207 of the handpiece 200.

The handpiece 300 differs from the handpiece 200 in that the rotary drive means operate by means of an air turbine and not by means of a micromotor.

More specifically, the handpiece 300 has a duct 354 passing successively through the counterweight 324, the piezoelectric motor 320, and the amplifier portion 326. The duct 354 leads to an air turbine 350 pivotally mounted in the housing 307 so as to be capable of driving an insert 310 in rotation when the insert is placed in the working position in the housing 307. This rotation is obtained under drive from a fluid under pressure (e.g. a stream of gas (such as air for example) that is delivered via the transmission duct 354 against the blades 352 of the gas turbine 350.

In this example, it is assumed that the air turbine 350 is fed with air.

The duct 354 may have two distinct passages, one for feeding gas to the turbine and the other for recovering gas. The design and the operation of an air turbine are well known to the person skilled in the art and are therefore not described in greater detail in this document.

The rotary drive means thus comprise the duct 354 and the air turbine 350.

In accordance with the invention, the rotary drive means in this example are also movable independently of the ultrasound piezoelectric transducer, with the ultrasound piezoelectric transducer being static relative to the handpiece 300.

It should be observed that the handpiece 300 presents a non-zero angle α between the distal portion 354b and the proximal portion 354a of the duct 354 (i.e. between the axes B and C) in a manner analogous to the angle formed by the proximal and distal portions 204a and 204b of the duct 204 in the handpiece 200. The presence of this non-zero angle α implies that the working head situated at the distal end of the handpiece is set back a little relative to the proximal portion of the handpiece that is to be held by the operator. This setback of the working head makes the handpiece more ergonomic in certain types of intervention, in particular in dentistry.

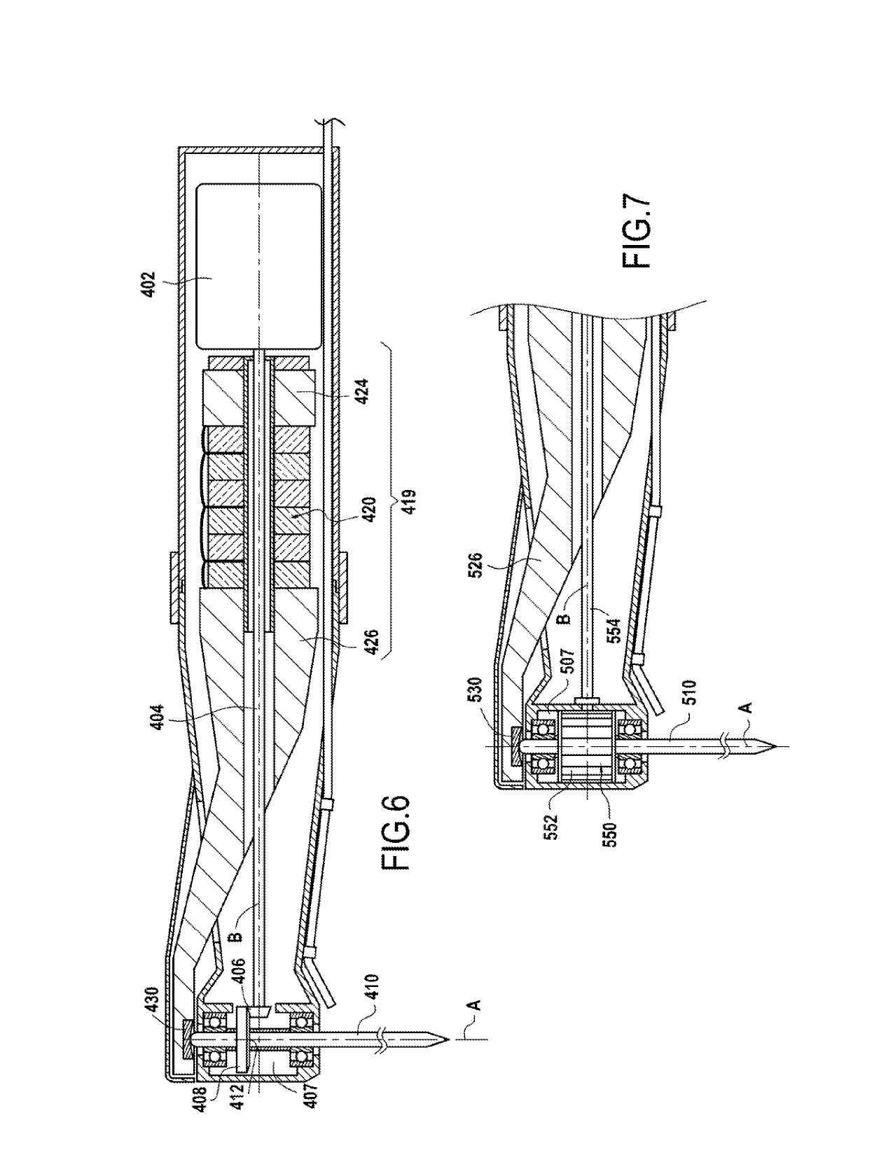

In a variant, the design of the handpieces 200 and 300 may be modified so that the angle α is zero, as shown respectively in FIGS. 6 and 7 (handpieces 400 and 500 respectively).

It should also be observed that the above-described handpieces present an angled shape. In the handpiece 200 (or 300), the axis of rotation A of the rotary drive means in the housing 207 (or 307) is perpendicular to the distal portion 204b (or 354b) of the transmission shaft 204 (or the duct 354). This angled configuration is also shown in the variants 400 and 500 shown in FIGS. 6 and 7.

As explained above, this angled shape provides advantages in terms of ergonomic access to the tissue that is to be treated, in particular in the context of dentistry. Nevertheless, this angled shape is not essential in the context of the invention.

By way of example, FIG. 8 shows a handpiece 600 constituting a variant of the handpiece 200 of FIG. 2.

The components of the handpiece 600 are for the most part identical to the corresponding components of the above-described handpiece 200. Unless otherwise specified, the components of the handpiece 600 present the same properties and perform the same functions as the corresponding components of the handpiece 200.

The ultrasound piezoelectric transducer 619 of the handpiece 600 comprises in particular a piezoelectric motor 620 arranged under mechanical stress between a counterweight 624 and an amplifier portion 626, the distal portion of the amplifier portion being present in a housing 607.

The rotary drive means of the handpiece 600 comprise a micromotor suitable for driving an insert 610 in rotation when the insert is installed in the working position in the housing 607. This rotary drive is achieved by means of a rotary drive shaft 608.

The embodiment of FIG. 8 differs mainly in that it is a handpiece that is straight and not angled as are the above-described handpieces.

In this example, the amplifier portion 626 is a body of revolution about the main axis of the handpiece 600 (i.e. the axis of rotation A of the insert in the housing 607). In this embodiment, the axis of the transmission shaft 604 coincides with the axis of rotation A of the rotary drive means in the housing 607.

Furthermore, the transmission shaft 604 in this example is provided at its distal end with a circular head 608 that is present in the housing 607. This head 608 is configured so that it is possible to secure the proximal end 610a of the insert 610 therein (e.g. by screwing it into a housing provided for this purpose).

The head 608 situated at the distal end of the transmission shaft 604 bears against the surface 627a of the distal end of the amplifier portion 626. Because of this physical contact at the margin of the housing 607 between the head 608 of the transmission shaft 604 and the surface 627a, the amplifier portion 626 is suitable for transmitting the ultrasound vibration generated by the piezoelectric motor 620 to the insert 610.

High levels of friction may occur at the interface between the head 608 (that is movable in rotation) and the distal end of the amplifier portion 626 (that is static). That is why, in this embodiment, the distal end of the amplifier portion is constituted by an annular part 627 having properties that enable good transmission of ultrasound vibration by friction to the insert 610 via the head 608.

It should be observed that in the above-described embodiments, the ultrasound piezoelectric transducer and the rotary drive means of the invention are completely independent from each other. Each of them co-operates with the insert in independent manner. In contrast, in the handpiece 600, the ultrasound piezoelectric transducer 619 transmits ultrasound waves into the housing 607 via the head 608 (which forms a portion of the rotary drive means).

Variants of the handpiece 600 are nevertheless possible in which the distal end of the amplifier portion comes directly into contact with the insert so that ultrasound vibration is transmitted directly. For example, it is possible to modify the shape of the head 608 so that a distal end portion of the amplifier portion 626 makes physical contact with the insert in operation.

By way of example, variants in a straight configuration of the handpiece 300 (using an air turbine) may also be envisaged in the ambit of the invention.

All of the above-envisaged variants present the same advantages as those set out for the handpiece 200.

The surgical handpiece of the invention finds a particular application in the treatment of biological tissues (enamel, dentine, mucous membrane, bone, . . . ) or of implanted prostheses such as implants, prostheses, or any other implantable active devices (heart valves, . . . ).

1-12. (canceled)

13. A surgical handpiece comprising:
a housing for receiving an insert;
an ultrasound piezoelectric transducer comprising a piezoelectric motor placed between a counterweight and a proximal end of an amplifier portion, the piezoelectric transducer being suitable for transmitting ultrasound waves from a distal end of said amplifier portion to said insert, the distal end being present in the housing; and
rotary drive means present in said housing for driving in rotation said insert;
the surgical handpiece being wherein the rotary drive means are movable independently of the ultrasound piezoelectric transducer, said ultrasound piezoelectric transducer being static relative to the handpiece.

14. A handpiece according to claim 13, wherein the rotary drive means comprise a micromotor connected to the housing via a transmission shaft.

15. A handpiece according to claim 14, wherein the axis of rotation of the rotary drive means in said housing is perpendicular to a distal portion of the transmission shaft.

16. A handpiece according to claim 14, wherein the axis of rotation of the rotary drive means in said housing is parallel to a distal portion of the transmission shaft.

17. A handpiece according to claim 16, wherein the ultrasound piezoelectric transducer is configured to transmit ultrasound waves to said housing via the rotary drive means.

18. A handpiece according to claim 13, wherein the rotary drive means comprise an air turbine and a transmission duct, said turbine being suitable for entering into rotation under the action of a stream of gas blown via said transmission duct.

19. A handpiece according to claim 18, wherein the axis of rotation of the rotary drive means in said housing is perpendicular to a distal portion of the transmission duct.

20. A handpiece according to claim 18, wherein the axis of rotation of the rotary drive means in said housing is parallel to a distal portion of the transmission duct.

21. A handpiece according to claim 13, wherein the ultrasound piezoelectric transducer includes a ceramic vibratory transmission element arranged at the distal end of the amplifier portion so as to transmit the ultrasound waves in said housing.

22. A handpiece according to claim 13, wherein the amplifier portion presents a constriction of position along the amplifier portion that is set in such a manner as to maximize the amplitude of the vibratory motion of the distal end of the amplifier portion.

23. A handpiece according to claim 13, also including an insert placed in part in the housing so as to be capable of co-operating with the ultrasound piezoelectric transducer and with the rotary drive means.

24. A handpiece according to claim 23, wherein the insert is arranged in such a manner that its proximal end is in contact with the distal end of the amplifier portion, and that the body of the insert is capable of being driven in rotation by the rotary drive means.
